(19) United States
(12) Patent Application Publication
(10) Pub. No.: US 2022/0125570 A1
(43) Pub. Date: Apr. 28, 2022

(54) URETERAL STENT AND RETRIEVAL MEANS

(71) Applicants: Hackensack University Medical Center, Hackensack, NJ (US); The Trustees of the Stevens Institute of Technology, Hoboken, NJ (US)

(72) Inventors: Michael Stifelman, Hackensack, NJ (US); Vikki Hazelwood, Hoboken, NJ (US); Becky Tucci, Hoboken, NJ (US); Saarah Mohammedi, Basking Ridge, NJ (US); Mithin Nair, Weehawken, NJ (US); Anthony Fragale, Jr., Ocean City, NJ (US); Alicia Galer, Whitesboro, NY (US)

(21) Appl. No.: 17/429,256

(22) PCT Filed: Feb. 5, 2020
(86) PCT No.: PCT/US2020/016889

§ 371 Date: Aug. 6, 2021

Related U.S. Application Data
(60) Provisional application No. 62/803,179, filed on Feb. 8, 2019.

(51) Int. Cl. A61F 2/04; A61F 2/95

(52) U.S. Cl. CPC A61F 2/04; A61F 2/95; A61F 2002/048; A61F 2220/0008; A61F 2210/009; A61F 2210/0076; A61F 2002/9528

(57) ABSTRACT

The described invention provides a ureteral stent device comprising a tubular structure comprising a proximal end and a distal end. The proximal end comprises a magnet, a magnetic tip, a magnetic coating, a magnetic metal, or a magnetic alloy. The distal end comprises a tapered tip and can form any one of a J-curl, a j-shape, or a pigtail loop for securing the distal end in a kidney. The proximal end is not secured in the bladder and terminates in the ureter.

### CROSS-REFERENCE TO RELATED APPLICATIONS

This application claims the benefit of priority to U.S. provisional application 62/803,179 (filed Feb. 8, 2019), which is incorporated by reference in its entirety.

### FIELD OF THE INVENTION

The described invention relates generally to medical devices, and more particularly to ureteral stents.

### BACKGROUND OF THE INVENTION

Ureteral Stent Structure and Function

The urinary tract system's main goal is to drain the body of urine. It consists of two kidneys, two ureters, a bladder and a urethra. Together the kidneys filter through 120-150 quarts of blood daily to produce 1-2 quarts of urine. The ureters are thin tubes of muscle, 3 to 4 mm in diameter and 25 to 30 cm in length, that act as a passageway for the urine from the kidneys to the bladder. The bladder is an expandable, muscular organ that stores the urine, up to 1.5-2 cups, until it can exit via the urethra during urination. (“The Urinary Tract & How It Works.” National Institute of Diabetes and Digestive and Kidney Diseases, U.S. Department of Health and Human Services, 1 Jan. 2014). This system can be compromised when there is a blockage in the ureter. Specifically, the ureters can become obstructed (blocked) due to injury or conditions which produce an interruption in urine flow. These blockages decrease urine flow to the bladder and increase the reflux of urine to the kidney. Conditions that can produce this interruption in urine flow include, but are not limited to, intrinsic obstruction of the ureter due to tumor growth, stricture or stones (such as kidney stones), compression of the ureter due to extrinsic tumor growth, stone fragment impaction in the ureter following extracorporeal shock wave lithotripsy, retroperitoneal fibrosis and ureteral procedures such as endopyelotomy and ureteroscopy.

A ureteral stricture is characterized by a narrowing of the ureteral lumen, which causes functional obstruction. The ureteral strictures can be classified as extrinsic or intrinsic, benign or malignant, and iatrogenic or non-iatrogenic.

Extrinsic malignant strictures include those caused by primary or metastatic cancer. This includes primary pelvic malignancies, particularly cancers of the cervix, prostate, bladder, and colon, which frequently cause extrinsic compression of the distal ureter. Retroperitoneal lymphadenopathy, caused by a wide range of malignancies, particularly lymphoma, testicular carcinoma, breast cancer, or prostate cancer, may cause proximal to midureteral obstruction. Extrinsic benign compression due to idiopathic retroperitoneal fibrosis (a slowly progressive disorder in which the ureters and other abdominal organs or vessels become blocked by a fibrous mass and inflammation in the back of the abdomen) may also cause unilateral or bilateral ureteral obstruction, leading to azotemia. Transitional cell carcinoma (“TCC”) may cause malignant intrinsic obstruction. Malignant ureteral obstruction is differentiated from benign ureteral obstruction by (1) the presence of an extrinsic mass on a CT scan or sonogram and (2) the appearance of the ureter on contrast-study images. Ureteral TCC may manifest as ureteral obstruction. Ureteral TCCs typically have an irregular mucosal pattern and are associated with dilatation of the ureter below the lesion (goblet sign). Benign strictures are usually smooth, without distal dilatation. In some cases, biopsy may be required to differentiate benign from malignant strictures. Biopsy samples can usually be collected ureteroscopically or with a fluoroscopically directed ureteral brush. Ureteral tumors can also be diagnosed during transureteral resection of the tumor with specialized ureteral resectoscopes. Benign intrinsic strictures can be congenital (e.g., congenital obstructing megaureter), iatrogenic, or non-iatrogenic (e.g., those that follow passage of calculi or chronic inflammatory ureteral involvement, such as tuberculosis and schistosomiasis). (Breyer, Benjamin Newell, M D, et al., “Ureteral Stricture” emedicine.medscape.com; Jan. 13, 2017.)

Kidney stones are small deposits that form in the kidneys ranging from the size of a grain of sand to the size of a pea, affecting about 11 percent of men and 6 percent of women. Once a person has experienced a kidney stone he/she has a 50 percent chance of developing another in the following 5 to 7 years. Smaller stones can pass on their own while larger ones need to be treated. The stones can cause sharp pains in the back, side, lower abdomen and groin, hematuria (blood in the urine), a constant need to urinate and pain during urination. Diagnosis is done by lab tests, abdominal x-rays or CT scans. Upon diagnosis, current treatment methods include lithotripsy to break a large stone into passable pieces, cystoscopy, ureteroscopy, and percutaneous nephrolithotomy. After these procedures or in cases of small stones, a short ureteral stent can be used to promote urine flow and open the ureter in order to pass the stones. (“Treatment for Kidney Stones.” National Institute of Diabetes and Digestive and Kidney Diseases, U.S. Department of Health and Human Services, 1 May 2017).

Kidney stones and tumors resulting from kidney and bladder cancer can cause blockages resulting in other dangerous symptoms like hydronephrosis. Hydronephrosis occurs when there is a buildup of urine or a reflux of urine in the kidneys due to a blockage or obstruction of a ureter. The renal pelvis portion of the kidney swells, causing pain in the in the side, back, abdomen or groin, pain during urination and increased urge to urinate. Hydronephrosis is caused by underlying illnesses such as kidney stones, scar tissue from previous surgery, tumors or cancer, or urinary tract infections. It can be diagnosed with x-ray, CT, MRI or cystoscopy. If treated early, hydronephrosis can be reversible, but if treated after 6 weeks permanent damage is done to the kidneys. (“Hydronephrosis.” National Kidney Foundation, 3 Feb. 2017). In order to treat hydronephrosis, the blockage in the ureter must be cleared and normal urine flow restored. Ureteral stents have proven to be very successful in doing this. In a study where 117 stents were inserted in patients for a dwelling time of 55 to 140 days to treat hydronephrosis, all patients showed improvement and reversal of hydronephrosis. The upper end of the dwelling time range consisted of patients with malignant strictures of the ureter. Kao, Ming Hong, and Chung Cheng Wang. (“The Efficacy and Safety of Ureteral Dilation and Long-Term Type Ureteral Stent for Patients with Ureteral Obstruction.” Urological Science, vol. 26, no. 1, Elsevier Taiwan LLC, 2015, pp. 65-68, doi:10.1016/j.urols.2014.06.001).

For the year 2018, there has been an estimated 81,190 and 63,340 new cases of bladder and kidney cancer in the United States respectively. 1 out of 27 males and 1 out of 89 females will be diagnosed with bladder cancer in their lifetime; 1 out of 48 males and 1 out of 83 females will be diagnosed with kidney cancer in their lifetime. (“Key Statistics About Kidney Cancer.” American Cancer Society, 1 Aug. 2017). Malignant tumors resulting from these cancers can cause ureteral obstruction leading to more serious side effects such as renal dysfunction, or kidney failure, and urosepsis, a severe infection in the urinary tract. If left untreated, patients with malignant ureteral obstruction have a survival rate of 3.7-15.3 months. The malignant ureteral obstructions can be treated with a long term ureteral stent, opening any blockages or strictures caused by the tumors. (Pavlovic, Kristina, et al. “Stents for Malignant Ureteral Obstruction.” Asian Journal of Urology, vol. 3, no. 3, Elsevier Ltd, 2016, pp. 142-49, doi:10.1016/j.ajur.2016.04.002).

A ureteral stent is a device designed to restore the flow of urine by creating a pathway for urinary drainage from the kidney to the bladder. To treat obstructions of urine flow, stents can avoid obstructions of the ureter (e.g., ureteral stones, ureteral tumors, etc.) that disrupt the flow of urine from the corresponding kidney to the bladder. In other surgical applications, ureteral stents can be used to enhance or protect the integrity of the ureter.

Generally, a ureteral stent is a thin, flexible tube threaded into the ureter, and is generally tubular in shape, terminating in two opposing ends: a distal end in the kidney and a proximal end in the bladder. FIG. 1 is an illustration of some available ureteral stents. Stent A is a 6-F polyurethane stent with standard proximal and distal pigtail loops to prevent migration and fenestrations along the entire shaft length. Stent B is a 7-F silicone stent with holes in the loops only. (Stents A and B are manufactured by and shown courtesy of Cook Urological, Spencer, Ind.) Stent C is a Flexima ureteral stent (Boston Scientific). This 10-F stent has a hydrophilic coating and holes in the loops only. Stent D is an Ultrathane Amplatz ureteral stent (Cook, Bloomington, Ind.). This 8.5-F polyurethane-latex stent has a hydrophilic coating and metal markers indicating shaft length (arrowheads). Stent E is a C-Flex Towers multilength stent (Cook Urological). This 6-F stent has a hydrophilic coating and ridges rather than fenestrations along its length to assist with urine flow. The internal lumen accommodates a 0.028-inch guide wire. There are multiple coils on each end of the stent (arrows), which give a usable shaft length of 22-32 cm. (Dyer, Raymond B., MD, “Complications of Ureteral Stent Placement” Radio Graphics, Volume 22-No. 5, September-October 2002: p. 1007.)

FIG. 2 is an illustration of a ureteral stent in the human body. (UrologySanAntonio.com, Accessed January 2019.) Both of the ends of the stent are coiled in a J-curl (also known as a j-shape or a pigtail loop). The J-curl reduces or prevents unintentional migration of the stent in the lumen of the ureter. Specifically, the J-curl in the kidney end is configured to retain the stent within the renal pelvis and to reduce or prevent unintended migration of the stent down the ureter. At the other end, the J-curl in the bladder end is positioned in the bladder, and is configured to reduce or prevent unintended stent migration upward toward the kidney.

Ureteral Stent Insertion Procedure

Image-guided ureteral stenting is most often performed by a specially trained interventional radiologist in an interventional radiology suite or occasionally in the operating room on an outpatient basis. However, some patients may require admission following the procedure. Prior to the procedure, an ultrasound, a computed tomography (“CT”) or magnetic resonance imaging (“MRI”) may be performed on the patient. The patient is then connected to monitors that track heart rate, blood pressure and pulse during the procedure. A nurse or technologist will insert an intravenous (“IV”) line into a vein in the hand or arm of the patient so that sedative medication can be given intravenously. Moderate sedation or general anesthesia may also be used. The interventional radiologist will use x-rays and/or ultrasound to locate the kidney and a needle will be inserted through the patient's skin into the kidneys. Contrast material will be injected through the needle. Using a fluoroscope, (an imaging device that uses x-rays to see structures, e.g., the ureter, on a fluorescent screen) a guide wire is inserted into the ureter. The stent is run over the guide wire and placed in its permanent position within the ureter. Ureteral stents are typically 10 to 15 inches long and less than a quarter inch thick. Once the stent has been placed, the guide wire is removed. As the guide wire is removed and exits the bladder, the proximal end of existing stents will coil into a pigtail spiral or a J-shape. Similarly, as the guide wire continues to be removed and is no longer in contact with the stent, the distal end of existing stents will lose its rigidity and also coil into a pigtail spiral or J-shape. The opening in the skin is then covered with a dressing. No sutures are needed.

Complications of Ureteral Stent Placement

A ureteral stent is not permanent and needs to be replaced periodically. The length of time a stent is left in place is referred to as an indwelling time, and is generally determined by the indication for placement and by physician experience. Indwelling times can range from a few days for relief of ureteral edema to the duration of the patient's life for maintenance of ureteral patency in case of obstruction from malignant disease. Manufacturers usually recommend exchange of stents at 3 to 6 month intervals, and studies have shown that the prevalence of complications increases with longer indwelling times. (Dyer, Raymond B., MD, “Complications of Ureteral Stent Placement” Radio Graphics, Volume 22-No. 5, September-October 2002: p. 1008.)

Placement of the stent and leaving the stent in place for too long can lead a multitude of discomforts and complications. Consequences of ureteral stent placement can include irritative bladder symptoms, which can be intolerable and require early stent removal. Suprapubic and loin pain are common occurrences in patients with stents. If the stent is too long, allowing the distal loop to impinge on the bladder base, direct irritation with consequent symptoms may occur. Vesicorenal reflux is inevitable with a patent stent in place. In a report of voiding cystourethrography (meaning a radiographic technique for visualizing a person's urethra and urinary bladder while the person urinates) performed in patients with stents, 80% of patients were shown to have reflux during the voiding stage of the examination. This is likely the cause of flank pain experienced during voiding by these patients. Despite improvements in biocompatible materials for stent construction, the epithelium of the renal collecting system, ureter, and bladder can react to the presence of the foreign body. Microscopic blood in urine (hematuria) can be seen in the majority of patients with a stent in place, and at times gross hematuria may develop. Pyuria as a response to the chronic irritation may also occur. (Dyer, Raymond B., MD, “Complications of Ureteral Stent Placement” Radio Graphics, Volume 22-No. 5, September-October 2002: p. 1008.)

Some complications of ureteral stents include, but are not limited to, urinary tract infections, malposition, migration, encrustation, and ureteral erosion or fistulation. Urinary tract infection may develop in the short term as a complication of instrumentation of a previously sterile urinary tract, or later as an extension of the underlying disease process. In most patients with ureteral obstruction, stent placement is performed with antibiotic prophylaxis, often as a single dose attendant to the procedure. In patients with a known urinary tract infection, stent insertion is delayed until appropriate treatment with culture-specific antibiotics allows urine sterilization. The presence of a foreign body may also lead to colonization of the urinary tract and, ultimately, of the stent itself. Eradication of these infections may eventually require exchange or removal of the stent. (Dyer, Raymond B., MD, “Complications of Ureteral Stent Placement” Radio Graphics, Volume 22-No. 5, September-October 2002: p. 1008-1009.)

Malposition is defined as an incorrect position of a stent relative to initial placement. Stents made of stiffer materials may penetrate the ureter, collecting system, and kidney parenchyma during placement, resulting in urinoma (a mass formed by encapsulated extravasated urine) or hematoma formation. Close observation of the configuration of the proximal stent loop may provide an indication of perforation of the renal pelvis. Reconstitution of the proximal and distal loops or curves of the stent depends on inherent memory in the construction material, after the guide wire or other delivery system is removed. If a stent of inadequate length is selected for insertion and an inadequate distal curl is left in the bladder, reconstitution of the upper curl over time may retract the distal stent tip into the ureter (“fish reeling”), thereby complicating retrieval.

An appropriate stent length is critical for the prevention of irritative voiding symptoms and malpositioning of the stent during insertion. Stent length may be based on operator experience, the measured length of the ureter as determined from imaging studies, the patient's body habitus, or use of the bent guide wire technique. Ideally, stents can be placed with fluoroscopic assistance and positioning problems identified and corrected at the time of insertion. It should be recognized, however, that a stent is not static within the urinary tract, and if the patient develops unusual or persistent symptoms, evaluation with conventional radiography may be warranted. (Dyer, Raymond B., MD, “Complications of Ureteral Stent Placement” Radio Graphics, Volume 22-No. 5, September-October 2002: p. 1009-1010.)

Migration of the stent within the urinary tract may also occur. The problem of downward migration of soft silicone tubing can be initially addressed by adding barbs along the shaft of the tube. Currently available, completely internalized stents combat migration with the presence of a proximal and distal J or pigtail. Nevertheless, peristalsis may discharge a stent (especially one constructed from softer materials) from the ureter. One can also speculate that the prevalence of this complication will increase with the use of stents coated with hydrophilic materials. Migration upward or downward can also occur as a result of late reconstitution of the retention curves as described earlier. (Dyer, Raymond B., MD, “Complications of Ureteral Stent Placement” Radio Graphics, Volume 22-No. 5, September-October 2002: p. 1011.)

Regarding encrustation (meaning a crust or calcification on the surface of a stent), no current stent is inert within the urinary tract. The presence of a stent provides a framework for deposition of urine constituents. Over time, this will occur with any stent. To prevent encrustation, dilution of the urine with high fluid intake and aggressive treatment of any urinary tract infection can be undertaken. Prevention of encrustation and possible stent occlusion is also one of the major indications for prophylactic exchange of ureteral stents as recommended by the manufacturer. The presence of lithogenic (promoting formation of calculi solid particles in the urinary system) urine, coupled with prolonged indwelling stent times, increases the risk of encrustation. A population of patients in whom stents were placed to assist with treatment of urinary tract stones, found that encrustation occurred in 9.2% of stents retrieved before 6 weeks, 47.5% of stents left in place for 6 to 12 weeks, and 76.3% of stents left in place longer than 12 weeks. Associated morbidity was found to be minimal if indwelling times did not exceed 6 weeks. However, close follow-up and monitoring of these patients is mandatory. Management of encrustation represents a continuum from therapeutic nuisance to major uro-logic intervention. Severe encrustation tends to preferentially deposit crystalline material at the renal or bladder end of the stent. This has been attributed to peristaltic “wiping” of the ureteral portion of the stent. Minimal stent encrustation may not prevent removal of the stent, and, if recognized, can be treated with extracorporeal shock wave lithotripsy (“ESWL”). Larger-volume encrustations must be dealt with before stent removal is attempted. (Dyer, Raymond B., MD, “Complications of Ureteral Stent Placement” Radio Graphics, Volume 22-No. 5, September-October 2002: p. 1015.)

A rare complication of ureteral stent placement is erosion of the stent into adjacent structures, especially the arterial system. A high degree of clinical suspicion is necessary if mortality from this complication is to be avoided. Intermittent hematuria in a patient with a stent is the usual reason for presentation, but massive hematuria to the point of circulatory collapse may occur and may be provoked by ureteral stent manipulation. Extensive pelvic surgery and irradiation appear to be contributing factors to the development of this complication because both may lead to ureteral ischemia (a decrease in smooth muscle perfusion). The chronic presence of a plastic stent within a ureter at risk, adjacent to a pulsating vascular structure (normal vessel or graft or pseudoaneurysm at the site of vascular repair), appears to produce the circumstances that are necessary for erosion to occur. Diagnosis by means of clinical examination or any imaging procedure may be difficult. Angio-graphic evaluation may be misdirected if the diagnosis is not considered. Appropriate diagnosis is integral to therapy, which may include open surgical techniques, interventional radiologic techniques, or a combination of the two. (Dyer, Raymond B., MD, “Complications of Ureteral Stent Placement” Radio Graphics, Volume 22-No. 5, September-October 2002: p. 1018-1019.)

Ureteral Stent Removal Procedure

There are two common ways to remove a ureteral stent. Prior to removal, if the stent has been in place for a while, an X-ray is used to see if the stent is smooth or if it has become lined with a crust because the crust can make it harder to remove the stent. First, some stents can be removed with an attached string that comes out of the urethra. In some cases, a patient will be able to remove the stent via the string at home, while in other cases, a doctor will remove the stent at their office or a hospital. Second, if the ureteral stent has no string, a removal procedure is required to remove the stent. The general procedure is conducted using a thin, lighted tube called a cystoscope, which requires general anesthesia. The doctor inserts the cystoscope into the urethra and on into the bladder. The scope allows the doctor to check areas of the bladder and urethra that usually don't show up well on X-rays. Next, the doctor will insert tiny tools through the cystoscope to remove the stent.

As discussed above, current ureteral stents may cause adverse effects including, but not limited to, discomfort, increased frequency to urinate, pain during urination, and bloody urine due to calcification of the J-curl in the bladder over time. Thus, there is a large unmet medical need for ureteral stents, which reduce complications caused by the “double J-curl” ureteral stent. The first issue with the “double J-curl” ureteral stent is grab ease and access to the stent upon removal. If the stent lies in the bladder upon removal, cystoscopy, endoscopy of the urinary bladder via the urethra, can be used. On the other hand, if the stent retrieves into the ureter due to reflux of urine, an outpatient procedure where a scope passes through the bladder and urethra and into the ureter known as ureteroscopy must be endured. (Nguyen, Mike, et al. “Patient Experiences And Preferences With Ureteral Stent Removal Techniques.” The Journal of Urology, vol. 191, no. 4, 1 Jan. 2015, doi:10.1016/j.juro.2014.02.697). Ureteroscopy should be avoided because the ureter is a difficult area for a surgeon to access due to the sheer small size of the ureter, only a 3-4 mm opening. Another key issue with the “double J-curl” ureteral stent is stent longevity. This is because calcium deposits form on the J curl located in the bladder which in turn causes adverse events such as discomfort, the frequency to urinate, pain during urination and bloody urine. (Lingeman, James E., et al. “Assessing the Impact of Ureteral Stent Design on Patient Comfort.” The Journal of Urology, vol. 181, no. 6, June 2009, pp. 2581-2587, doi:10.1016/j.juro.2009.02.019). Grade 3 and grade 4 adverse events may require the stent to be removed prior to the designated time allotment, allowing only short-term use of the ureteral stent Dakkak, Y., et al. “Management of Encrusted Ureteral Stents.” African Journal of Urology, vol. 18, no. 3, September 2012, pp. 131-134, doi:10.1016/j.afju.2012.08.013).

As such, a need exists for an ureteral stent that can reduce these adverse effects, facilitate stent removal and improve that patient's quality of life.

The present disclosure includes a device designed to treat obstruction of urine flow from the kidney to the bladder for patients. The standard of care provided by urologists causes adverse events including, but not limited to, discomfort in the bladder, the frequency to urinate, and pain during urination related to the encrustation of the J-curl in the bladder, which impairs quality of life. A described invention eliminates the distal J-curl and replaces it with electromagnetic technology will eliminate ureteroscopy, increase stent longevity, and provide optimal comfort for long-term use. In turn, the described invention may elicit a decrease in hospital visits, reducing patient spending.

### SUMMARY OF THE INVENTION

According to one aspect, the described invention provides an ureteral stent device comprising a tubular structure comprising a proximal end and a distal end. The proximal end comprises a magnet, a magnetic tip, a magnetic coating, a magnetic metal, or a magnetic alloy. The distal end comprises a tapered tip and can form any one of a J-curl, a j-shape, or a pigtail loop for securing the distal end in a kidney. Further, the proximal end is not secured in the bladder and terminates in the ureter.

According to another aspect, the described invention provides an ureteral stent device comprising a tubular structure comprising a proximal end, a distal end, a cap, and an attachment device. The cap is attached to the proximal end via the attachment device. The distal end comprises a tapered tip and can form any one of a J-curl, a j-shape, or a pigtail loop for securing the distal end in a kidney. Further, the distal end is not secured in the bladder and terminates in the ureter.

According to another aspect, the described invention provides a ureteral stent removal device comprising a magnetic end, wherein the magnetic end comprises a coating to prevent encrustation. The magnetic end of the stent retrieval tool can be inserted into the urethra and maneuvered to make contact with a magnet at the proximal end of the ureteral stent device. Once contact is made, the stent retrieval tool can be used to withdraw the ureteral stent device from the ureter via the urethra.

According to another aspect, the stent retrieval tool utilizes electromagnetic technology. The stent retrieval tool can comprise a metallic rod, or any other suitable rod capable of being magnetized, fixated to a ureteral catheter. The stent retrieval tool can further include a power source to magnetize the rod so that the rod can attract the proximal end of the ureteral stent device.

These and other advantages of the invention will be apparent to those of ordinary skill in the art by reference to the following detailed description and the accompanying drawings.

In the various views of the drawings, like reference characters designate like or similar parts.

### BRIEF DESCRIPTION OF THE DRAWINGS

FIG. 1 shows an illustrative view of ureteral stents; (from Dyer, Raymond B., MD, “Complications of Ureteral Stent Placement” Radio Graphics, Volume 22-No. 5, September-October 2002: p. 1007);

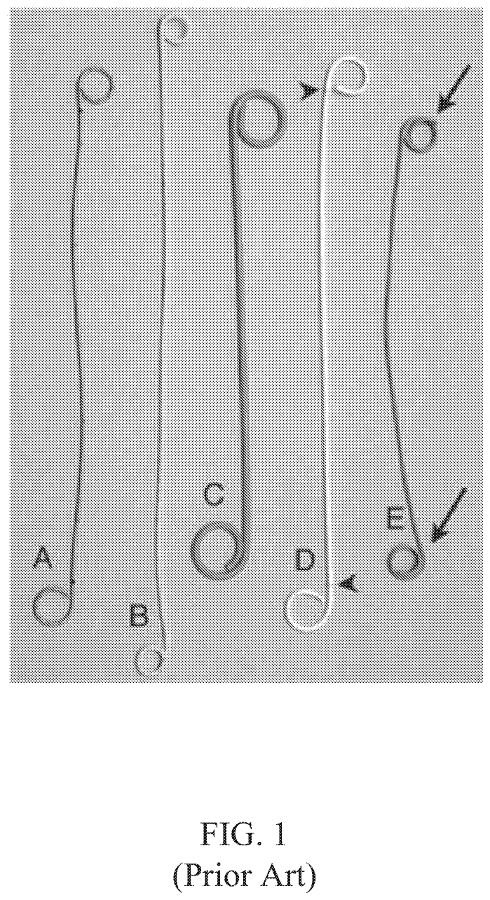

FIG. 2 shows an illustrative view of a ureteral stent in the human body; (from UrologySanAntonio.com, Accessed January 2019);

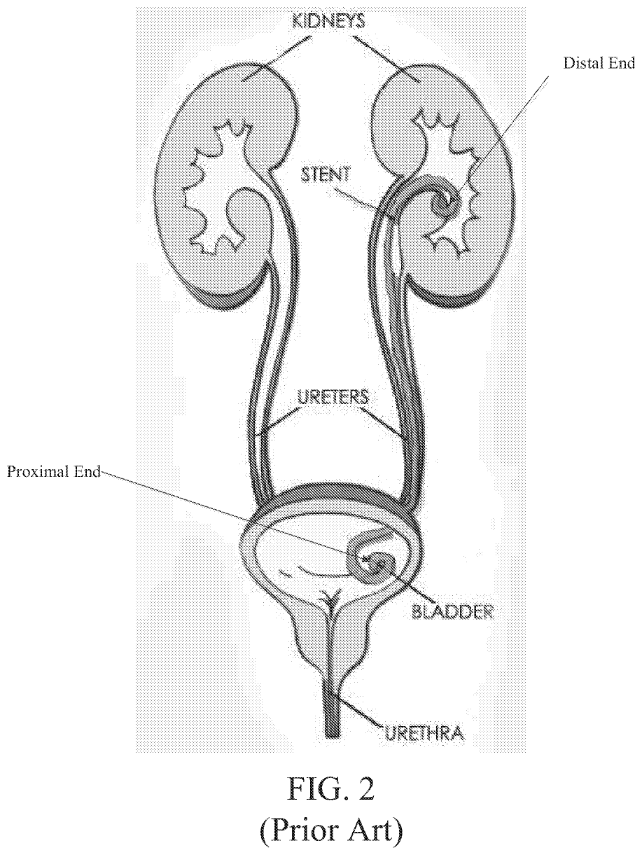

FIGS. 3A-3B show an illustrative embodiment of the ureteral stent device 300 of the present disclosure;

FIG. 4 shows an illustrative embodiment of the ureteral stent device 300 of the present disclosure in a human body.

FIG. 5 shows an illustration of the ureteral stent device 300 of the present disclosure in comparison with a traditional “double J-curl” ureteral stent 400;

FIG. 6 shows an illustrative view of an electromagnet of the present disclosure comprising copper wire wound down the long axis of a steel rod;

FIG. 7 shows equations for force excerted by a magnetic field;

FIG. 8 shows equations for a closed magnetic circuit;

FIG. 9 is a graph showing the force required to separate the ureteral stent device 300 and a stent retrieval tool, of the present disclosure;

FIG. 10 is a graph showing projected results for a flow rate test using the ureteral stent device 300, of the present disclosure;

FIG. 11 is a table showing the magnetic properties of cobalt-chromium;

FIG. 12 is a illustrative embodiment of the a cobalt-chromium proximal end of the present disclosure;

FIG. 13 shows an illustrative embodiment of the ureteral stent device 700 of the present disclosure; and

FIGS. 14A and 14B show illustration of a closer view of the cap 716 and the attachment device 718 of the ureteral stent device 700.

### DETAILED DESCRIPTION OF THE INVENTION

### Glossary

The term “proximal” as used herein, refers to the state of being situated next to or nearest the point of attachment or origin.

The term “distal” as used herein, refers to the state of being situated away from the point of attachment or origin

The term “tapered” as used herein, means being diminished or reduced in thickness towards the end.

The term “biocompatible” as used herein, means causing no clinically relevant tissue irritation, injury, toxic reaction, or immunologic reaction to human tissue based on a clinical risk/benefit assessment.

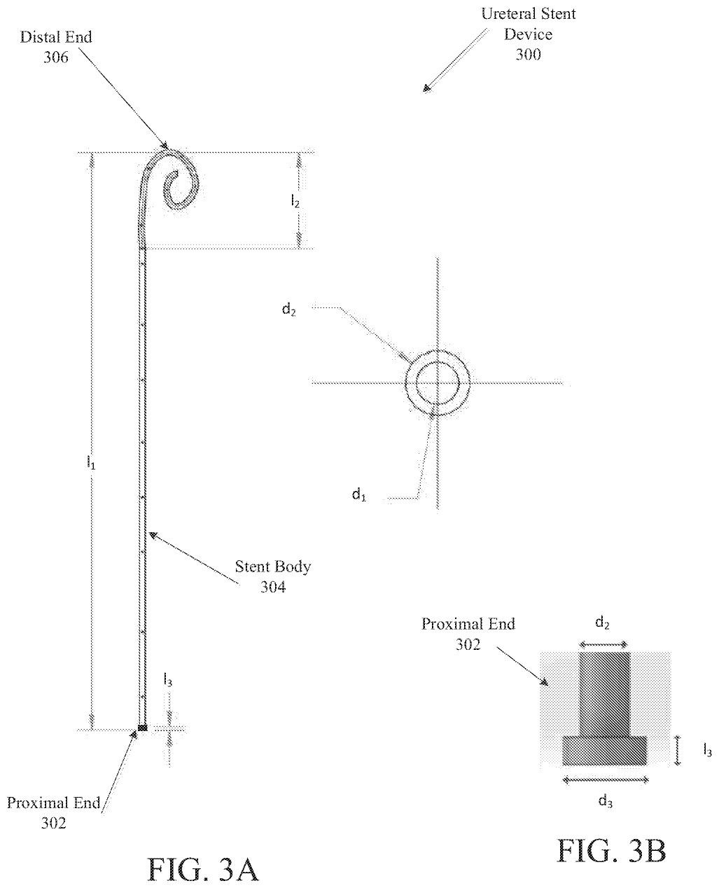

The present disclosure relates to a ureteral stent device and removal means, as discussed in detail below in connection with FIGS. 3-14.

FIG. 3A shows an exemplary and non-limiting example of an ureteral stent device 300 of the described invention. The ureteral stent device 300 of the described invention comprises a proximal end 302, a stent body 304, and a distal end 306. The stent body 304 comprises a tubular structure comprising an inner diameter d₁ and an outer diameter d₂, making the stent body 304 hollow. The hollow body allows urine to flow through ureteral stent device 300. The proximal end 302 comprises a magnet, a magnetic tip, a magnetic coating, a magnetic metal, a magnetic alloy, etc. It should be understood that the proximal end 302 is hollow to allow urine to flow through the ureteral stent device 300. By way of example, the magnet is a neodymium iron baron magnet. However, those skilled in the art would understand that the proximal end 302 can comprise any other biocompatible magnet. The magnet can be coated with a biocompatible coating, such as, for example, Teflon, titanium, or any other coating effective to prevent or reduce encrustation (e.g., heparin, phosphorylcholine, antibiotic, carbon, hyaluronic acid, triclosan, silver, gendine, chitosan, salicylic acid, hydrogel, etc.). The magnet can be fixated to the proximal end 302. In an embodiment, the magnet is fixated to the proximal end using a waterproof adhesive, such as but not limited to, polyurethane glue. Those skilled in the art would understand that other fixating methods can be used, such, as but not limited to, a clip mechanism, a screwing mechanism, welding, soldering, taping, inserting, etc.

The ureteral stent device 300 is of length l₁, with the distal end 306 curled. The distal end 306, when curled, is of length l₂. According to some embodiments, the length of the stent body 304 and the proximal end 302 (i.e. l₁-l₂) does not exceed the length of the human ureter in order to keep material out of the bladder. The average length of a human ureter is generally 25 cm-30 cm. According to some embodiments, the length of the stent body 304 and the proximal end 302 ranges from 1 cm to 50 cm. According to some embodiments, the length of the stent body 304 and the proximal end 302 is at least 1 cm. According to some embodiments, the length of the stent body 304 and the proximal end 302 is at least 2 cm. According to some embodiments, the length of the stent body 304 and the proximal end 302 is at least 3 cm. According to some embodiments, the length of the stent body 304 and the proximal end 302 is at least 4 cm. According to some embodiments, the length of the stent body 304 and the proximal end 302 is at least 5 cm. According to some embodiments, the length of the stent body 304 and the proximal end 302 is at least 6 cm. According to some embodiments, the length of the stent body 304 and the proximal end 302 is at least 7 cm. According to some embodiments, the length of the stent body 304 and the proximal end 302 is at least 8 cm. According to some embodiments, the length of the stent body 304 and the proximal end 302 is at least 9 cm. According to some embodiments, the length of the stent body 304 and the proximal end 302 is at least 10 cm. According to some embodiments, the length of the stent body 304 and the proximal end 302 is at least 11 cm. According to some embodiments, the length of the stent body 304 and the proximal end 302 is at least 12 cm. According to some embodiments, the length of the stent body 304 and the proximal end 302 is at least 13 cm. According to some embodiments, the length of the stent body 304 and the proximal end 302 is at least 14 cm. According to some embodiments, the length of the stent body 304 and the proximal end 302 is at least 15 cm. According to some embodiments, the length of the stent body 304 and the proximal end 302 is at least 16 cm. According to some embodiments, the length of the stent body 304 and the proximal end 302 is at least 17 cm. According to some embodiments, the length of the stent body 304 and the proximal end 302 is at least 18 cm. According to some embodiments, the length of the stent body 304 and the proximal end 302 is at least 19 cm. According to some embodiments, the length of the stent body 304 and the proximal end 302 is at least 20 cm. According to some embodiments, the length of the stent body 304 and the proximal end 302 is at least 21 cm. According to some embodiments, the length of the stent body 304 and the proximal end 302 is at least 22 cm. According to some embodiments, the length of the stent body 304 and the proximal end 302 is at least 23 cm. According to some embodiments, the length of the stent body 304 and the proximal end 302 is at least 24 cm. According to some embodiments, the length of the stent body 304 and the proximal end 302 is at least 25 cm. According to some embodiments, the length of the stent body 304 and the proximal end 302 is at least 26 cm. According to some embodiments, the length of the stent body 304 and the proximal end 302 is at least 27 cm. According to some embodiments, the length of the stent body 304 and the proximal end 302 is at least 28 cm. According to some embodiments, the length of the stent body 304 and the proximal end 302 is at least 29 cm. According to some embodiments, the length of the stent body 304 and the proximal end 302 is at least 30 cm. According to some embodiments, the length of the stent body 304 and the proximal end 302 is at least 31 cm. According to some embodiments, the length of the stent body 304 and the proximal end 302 is at least 32 cm. According to some embodiments, the length of the stent body 304 and the proximal end 302 is at least 33 cm. According to some embodiments, the length of the stent body 304 and the proximal end 302 is at least 34 cm. According to some embodiments, the length of the stent body 304 and the proximal end 302 is at least 35 cm. According to some embodiments, the length of the stent body 304 and the proximal end 302 is at least 36 cm. According to some embodiments, the length of the stent body 304 and the proximal end 302 is at least 37 cm. According to some embodiments, the length of the stent body 304 and the proximal end 302 is at least 38 cm. According to some embodiments, the length of the stent body 304 and the proximal end 302 is at least 39 cm. According to some embodiments, the length of the stent body 304 and the proximal end 302 is at least 40 cm. According to some embodiments, the length of the stent body 304 and the proximal end 302 is at least 41 cm. According to some embodiments, the length of the stent body 304 and the proximal end 302 is at least 42 cm. According to some embodiments, the length of the stent body 304 and the proximal end 302 is at least 43 cm. According to some embodiments, the length of the stent body 304 and the proximal end 302 is at least 44 cm. According to some embodiments, the length of the stent body 304 and the proximal end 302 is at least 45 cm. According to some embodiments, the length of the stent body 304 and the proximal end 302 is at least 46 cm. According to some embodiments, the length of the stent body 304 and the proximal end 302 is at least 47 cm. According to some embodiments, the length of the stent body 304 and the proximal end 302 is at least 48 cm. According to some embodiments, the length of the stent body 304 and the proximal end 302 is at least 49 cm. According to some embodiments, the length of the stent body 304 and the proximal end 302 is at least 50 cm.

The minimum diameter of a human ureter is generally 3 mm, but can exceed this minimum diameter. (Zelenko, N, et al. “Normal Ureter Size on Unenhanced Helical CT.” Current Neurology and Neuroscience Reports, U.S. National Library of Medicine, April 2004). For example, the diameter of the proximal end 302 (d₃) and the diameter of the stent body 304 (d₂) does not exceed a patient's ureter diameter.

According to some embodiments, the outer diameter of the proximal end 302 (d₂) ranges from 1.0 mm to 5.0 mm. According to some embodiments, d₂ is at least 1.0 mm. According to some embodiments, d₂ is at least 1.1 mm. According to some embodiments, d₂ is at least 1.2 mm. According to some embodiments, d₂ is at least 1.3 mm. According to some embodiments, d₂ is at least 1.4 mm. According to some embodiments, d₂ is at least 1.5 mm. According to some embodiments, d₂ is at least 1.6 mm. According to some embodiments, d₂ is at least 1.7 mm. According to some embodiments, d₂ is at least 1.8 mm. According to some embodiments, d₂ is at least 1.9 mm. According to some embodiments, d₂ is at least 2.0 mm. According to some embodiments, d₂ is at least 2.0 mm. According to some embodiments, d₂ is at least 2.1 mm. According to some embodiments, d₂ is at least 2.2 mm. According to some embodiments, d₂ is at least 2.3 mm. According to some embodiments, d₂ is at least 2.4 mm. According to some embodiments, d₂ is at least 2.5 mm. According to some embodiments, d₂ is at least 2.6 mm. According to some embodiments, d₂ is at least 2.7 mm. According to some embodiments, d₂ is at least 2.8 mm. According to some embodiments, d₂ is at least 2.9 mm. According to some embodiments, d₂ is at least 3.0 mm. According to some embodiments, d₂ is at least 3.1 mm. According to some embodiments, d₂ is at least 3.2 mm. According to some embodiments, d₂ is at least 3.3 mm. According to some embodiments, d₂ is at least 3.4 mm. According to some embodiments, d₂ is at least 3.5 mm. According to some embodiments, d₂ is at least 3.6 mm. According to some embodiments, d₂ is at least 3.7 mm. According to some embodiments, d₂ is at least 3.8 mm. According to some embodiments, d₂ is at least 3.9 mm. According to some embodiments, d₂ is at least 4.0 mm. According to some embodiments, d₂ is at least 4.1 mm. According to some embodiments, d₂ is at least 4.2 mm. According to some embodiments, d₂ is at least 4.3 mm. According to some embodiments, d₂ is at least 4.4 mm. According to some embodiments, d₂ is at least 4.5 mm. According to some embodiments, d₂ is at least 4.6 mm. According to some embodiments, d₂ is at least 4.7 mm. According to some embodiments, d₂ is at least 4.8 mm. According to some embodiments, d₂ is at least 4.9 mm. According to some embodiments, d₂ is at least 5.0 mm.

According to some embodiments, the inner diameter d₁ of the proximal end 302 (d₁) ranges from 0.1 mm to 4.0 mm. According to some embodiments, d₁ is at least 0.1 mm. According to some embodiments, d₁ is at least 0.2 mm. According to some embodiments, d₁ is at least 0.3 mm. According to some embodiments, d₁ is at least 0.4 mm. According to some embodiments, d₁ is at least 0.5 mm. According to some embodiments, d₁ is at least 0.6 mm. According to some embodiments, d₁ is at least 0.7 mm. According to some embodiments, d₁ is at least 0.8 mm. According to some embodiments, d₁ is at least 0.9 mm. According to some embodiments, d₁ is at least 1.0 mm. According to some embodiments, d₁ is at least 1.1 mm. According to some embodiments, d₁ is at least 1.2 mm. According to some embodiments, d₁ is at least 1.3 mm. According to some embodiments, d₁ is at least 1.4 mm. According to some embodiments, d₁ is at least 1.5 mm. According to some embodiments, d₁ is at least 1.6 mm. According to some embodiments, d₁ is at least 1.7 mm. According to some embodiments, d₁ is at least 1.8 mm. According to some embodiments, d₁ is at least 1.9 mm. According to some embodiments, d₁ is at least 2.0 mm. According to some embodiments, d₁ is at least 2.0 mm. According to some embodiments, d₁ is at least 2.1 mm. According to some embodiments, d₁ is at least 2.2 mm. According to some embodiments, d₁ is at least 2.3 mm. According to some embodiments, d₁ is at least 2.4 mm. According to some embodiments, d₁ is at least 2.5 mm. According to some embodiments, d₁ is at least 2.6 mm. According to some embodiments, d₁ is at least 2.7 mm. According to some embodiments, d₁ is at least 2.8 mm. According to some embodiments, d₁ is at least 2.9 mm. According to some embodiments, d₁ is at least 3.0 mm. According to some embodiments, d₁ is at least 3.1 mm. According to some embodiments, d₁ is at least 3.2 mm. According to some embodiments, d₁ is at least 3.3 mm. According to some embodiments, d₁ is at least 3.4 mm. According to some embodiments, d₁ is at least 3.5 mm. According to some embodiments, d₁ is at least 3.6 mm. According to some embodiments, d₁ is at least 3.7 mm. According to some embodiments, d₁ is at least 3.8 mm. According to some embodiments, d₁ is at least 3.9 mm. According to some embodiments, d₁ is at least 4.0 mm.

FIG. 3B shows exemplary and non-limiting examples of the proximal end 302 of the ureteral stent device 300. The proximal end 302 comprises a cylindrical magnet comprising a diameter d₃ and a length of l₃. According to some embodiments, d₃ ranges from 1.0 mm to 5.0 mm. According to some embodiments, d₃ is at least 1.0 mm. According to some embodiments, d₃ is at least 1.1 mm. According to some embodiments, d₃ is at least 1.2 mm. According to some embodiments, d₃ is at least 1.3 mm. According to some embodiments, d₃ is at least 1.4 mm. According to some embodiments, d₃ is at least 1.5 mm. According to some embodiments, d₃ is at least 1.6 mm. According to some embodiments, d₃ is at least 1.7 mm. According to some embodiments, d₃ is at least 1.8 mm. According to some embodiments, d₃ is at least 1.9 mm. According to some embodiments, d₃ is at least 2.0 mm. According to some embodiments, d₃ is at least 2.0 mm. According to some embodiments, d₃ is at least 2.1 mm. According to some embodiments, d₃ is at least 2.2 mm. According to some embodiments, d₃ is at least 2.3 mm. According to some embodiments, d₃ is at least 2.4 mm. According to some embodiments, d₃ is at least 2.5 mm. According to some embodiments, d₃ is at least 2.6 mm. According to some embodiments, d₃ is at least 2.7 mm. According to some embodiments, d₃ is at least 2.8 mm. According to some embodiments, d₃ is at least 2.9 mm. According to some embodiments, d₃ is at least 3.0 mm. According to some embodiments, d₃ is at least 3.1 mm. According to some embodiments, d₃ is at least 3.2 mm. According to some embodiments, d₃ is at least 3.3 mm. According to some embodiments, d₃ is at least 3.4 mm. According to some embodiments, d₃ is at least 3.5 mm. According to some embodiments, d₃ is at least 3.6 mm. According to some embodiments, d₃ is at least 3.7 mm. According to some embodiments, d₃ is at least 3.8 mm. According to some embodiments, d₃ is at least 3.9 mm. According to some embodiments, d₃ is at least 4.0 mm. According to some embodiments, d₃ is at least 4.1 mm. According to some embodiments, d₃ is at least 4.2 mm. According to some embodiments, d₃ is at least 4.3 mm. According to some embodiments, d₃ is at least 4.4 mm. According to some embodiments, d₃ is at least 4.5 mm. According to some embodiments, d₃ is at least 4.6 mm. According to some embodiments, d₃ is at least 4.7 mm. According to some embodiments, d₃ is at least 4.8 mm. According to some embodiments, d₃ is at least 4.9 mm. According to some embodiments, d₃ is at least 5.0 mm.

According to some embodiments, l₃ ranges from 1.0 mm to 5.0 mm. According to some embodiments, l₃ is at least 1.0 mm. According to some embodiments, l₃ is at least 1.1 mm. According to some embodiments, l₃ is at least 1.2 mm. According to some embodiments, l₃ is at least 1.3 mm. According to some embodiments, l₃ is at least 1.4 mm. According to some embodiments, l₃ is at least 1.5 mm. According to some embodiments, l₃ is at least 1.6 mm. According to some embodiments, l₃ is at least 1.7 mm. According to some embodiments, l₃ is at least 1.8 mm. According to some embodiments, l₃ is at least 1.9 mm. According to some embodiments, l₃ is at least 2.0 mm. According to some embodiments, l₃ is at least 2.0 mm. According to some embodiments, l₃ is at least 2.1 mm. According to some embodiments, l₃ is at least 2.2 mm. According to some embodiments, l₃ is at least 2.3 mm. According to some embodiments, l₃ is at least 2.4 mm. According to some embodiments, l₃ is at least 2.5 mm. According to some embodiments, l₃ is at least 2.6 mm. According to some embodiments, l₃ is at least 2.7 mm. According to some embodiments, l₃ is at least 2.8 mm. According to some embodiments, l₃ is at least 2.9 mm. According to some embodiments, l₃ is at least 3.0 mm. According to some embodiments, l₃ is at least 3.1 mm. According to some embodiments, l₃ is at least 3.2 mm. According to some embodiments, l₃ is at least 3.3 mm. According to some embodiments, l₃ is at least 3.4 mm. According to some embodiments, l₃ is at least 3.5 mm. According to some embodiments, l₃ is at least 3.6 mm. According to some embodiments, l₃ is at least 3.7 mm. According to some embodiments, l₃ is at least 3.8 mm. According to some embodiments, l₃ is at least 3.9 mm. According to some embodiments, l₃ is at least 4.0 mm. According to some embodiments, l₃ is at least 4.1 mm. According to some embodiments, l₃ is at least 4.2 mm. According to some embodiments, l₃ is at least 4.3 mm. According to some embodiments, l₃ is at least 4.4 mm. According to some embodiments, l₃ is at least 4.5 mm. According to some embodiments, l₃ is at least 4.6 mm. According to some embodiments, l₃ is at least 4.7 mm. According to some embodiments, l₃ is at least 4.8 mm. According to some embodiments, l₃ is at least 4.9 mm. According to some embodiments, l₃ is at least 5.0 mm. It should be understood that, according to some embodiments, l₃ ranges from 0.1 mm to 1.0 mm, and according to other embodiments, l₃ ranges from 5 mm to 50 mm.

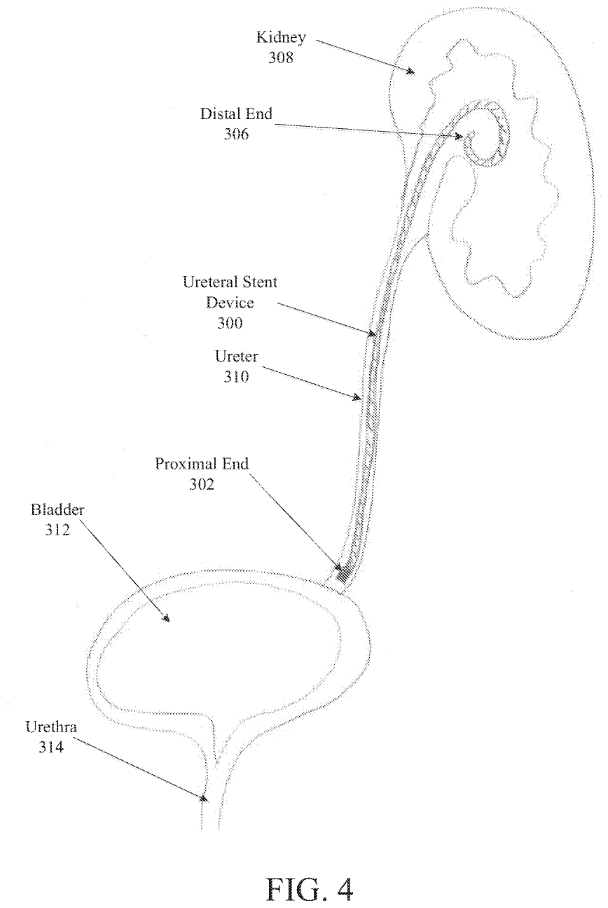

FIG. 4 shows an exemplary and non-limiting example of a ureteral stent device 300 of the described invention in a human body. Specifically, FIG. 4 shows the proximal end 302, the stent body 304, and the distal end 306, a kidney 308, a ureter 310, a bladder 312, and a urethra 314. The distal end 306 comprises a tip and can form any one of a J-curl, a j-shape, a pigtail loop, or any other type of curl capable of securing the distal end 306 in the kidney 308. The tip can be tapered. The kidney 308 is used to show a general relation between the kidney entrance and the distal end 306, and is used for illustrative purposes only. Similarly, the bladder 312 is used to show a general relation between the bladder and the proximal end 302, and is used for illustrative purposes only.

In this embodiment, the proximal end 302 does not enter the bladder 312, and terminates in the ureter 310. This is solely by way of example. In other embodiments, the proximal end 302 can terminal in the bladder 312, or anywhere else along the ureter 310.

It should be appreciated by those skilled in the art that the proximal end 302 does not comprise a J-curl, and terminates in the ureter 310 or near the entrance of the bladder 312. This reduces patient discomfort, bladder irritation, and encrustation when compared to a more traditional “double J-curl” ureteral stent because the ureteral stent device 300 eliminates material residing in the bladder 312.

Insertion of the ureteral stent device 300 into the human body can follow current methods and standards required by the FDA. The procedure entails utilizing a guidewire inserted into the urethra 314 which travels to the kidney 308, monitored via fluoroscopy. The stent is then advanced over the guidewire using a stent pusher until the distal J-curl is completely in the kidney. The positioning of the ureteral stent device 300 is then confirmed using fluoroscopy. Once the ureteral stent device 300 is correctly positioned, the guidewire is removed, completing the insertion procedure.

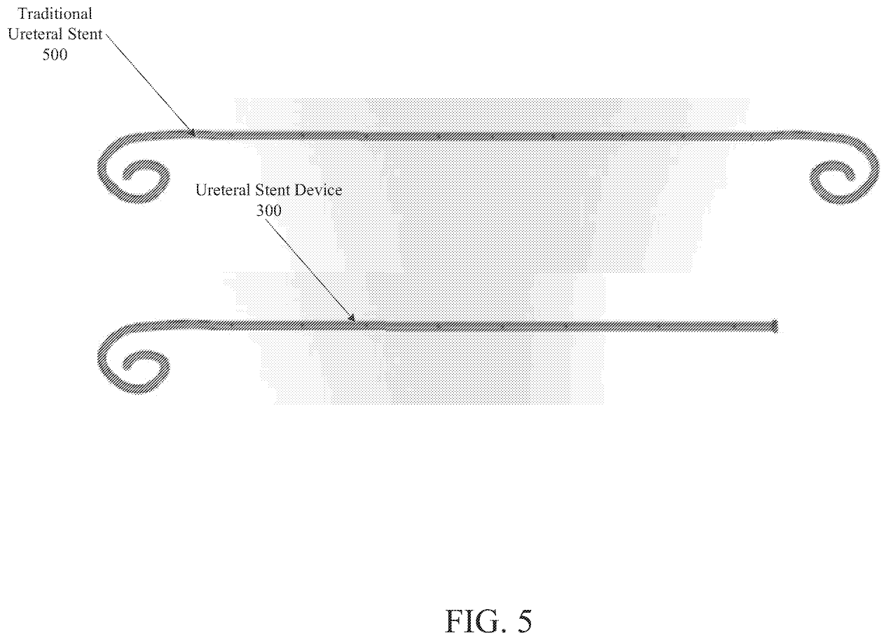

FIG. 5 shows an illustration of the ureteral stent device 300 in comparison with a traditional “double J-curl” ureteral stent 500. Specifically, the traditional “double J-curl” ureteral stent 500 comprises a J-curl at each end, while the ureteral stent device 300 of the disclosed invention comprises a J-curl on one end, and a magnet on the other end.

The ureteral stent device 300 can be retrieved by employing a stent retrieval tool. In one example, the stent retrieval tool can comprise a magnet at one end, where a polarity of the magnet at the end of the stent retrieval tool is reactive to the polarity of the magnetic tip of the ureteral stent device 300, meaning a pole of a magnet in space with relatively more electrons is said to have negative polarity, while the pole with relatively fewer electrons is said to have positive polarity. The pole with negative polarity of the proximal end 302 is attracted to the pole with positive polarity of the stent retrieval tool, or vice versa. The magnet can comprise a coating. By way of example, the magnet at the end of the stent retrieval tool can comprise a neodymium iron baron magnet or any other biocompatible magnet and the coating can comprise Teflon, titanium, or any other coating effective to prevent or reduce encrustation. The magnetic end of the stent retrieval tool can be inserted into the urethra 314 and maneuvered to make contact (meaning state or condition of touching or being in immediate proximity) with the magnet at the proximal end 302. Once contact is made, the stent retrieval tool can be used to withdraw the ureteral stent device 300 from the ureter via the urethra 314.

In another example, the stent retrieval tool utilizes electromagnetic technology. The stent retrieval tool can comprise a metallic rod (e.g., steel, stainless steel, iron, nickel, cobalt, rare earth metals, etc.), or any other suitable rod capable of being magnetized, fixated to a ureteral catheter. The stent retrieval tool can be fixated to the ureteral catheter via an adhesive (e.g., polyurethane glue), a clip mechanism, a screwing mechanism, welding, soldering, taping, inserting, etc. The rod can be electromagnetically charged and contacted to the proximal end of the ureteral stent device 300 to remove the device 300. It is noted that pinching of the ureter wall will not occur when the diameter of the rod is less than the diameter of magnet. According to some embodiments, the diameter of the rod ranges from 1.0 mm to 5.0 mm. According to some embodiments, the diameter of the rod is at least 1.0 mm. According to some embodiments, the diameter of the rod is at least 1.1 mm. According to some embodiments, the diameter of the rod is at least 1.2 mm. According to some embodiments, the diameter of the rod is at least 1.3 mm. According to some embodiments, the diameter of the rod is at least 1.4 mm. According to some embodiments, the diameter of the rod is at least 1.5 mm. According to some embodiments, the diameter of the rod is at least 1.6 mm. According to some embodiments, the diameter of the rod is at least 1.7 mm. According to some embodiments, the diameter of the rod is at least 1.8 mm. According to some embodiments, the diameter of the rod is at least 1.9 mm. According to some embodiments, the diameter of the rod is at least 2.0 mm. According to some embodiments, the diameter of the rod is at least 2.0 mm. According to some embodiments, the diameter of the rod is at least 2.1 mm. According to some embodiments, the diameter of the rod is at least 2.2 mm. According to some embodiments, the diameter of the rod is at least 2.3 mm. According to some embodiments, the diameter of the rod is at least 2.4 mm. According to some embodiments, the diameter of the rod is at least 2.5 mm. According to some embodiments, the diameter of the rod is at least 2.6 mm. According to some embodiments, the diameter of the rod is at least 2.7 mm. According to some embodiments, the diameter of the rod is at least 2.8 mm. According to some embodiments, the diameter of the rod is at least 2.9 mm. According to some embodiments, the diameter of the rod is at least 3.0 mm. According to some embodiments, the diameter of the rod is at least 3.1 mm. According to some embodiments, the diameter of the rod is at least 3.2 mm. According to some embodiments, the diameter of the rod is at least 3.3 mm. According to some embodiments, the diameter of the rod is at least 3.4 mm. According to some embodiments, the diameter of the rod is at least 3.5 mm. According to some embodiments, the diameter of the rod is at least 3.6 mm. According to some embodiments, the diameter of the rod is at least 3.7 mm. According to some embodiments, the diameter of the rod is at least 3.8 mm. According to some embodiments, the diameter of the rod is at least 3.9 mm. According to some embodiments, the diameter of the rod is at least 4.0 mm. According to some embodiments, the diameter of the rod is at least 4.1 mm. According to some embodiments, the diameter of the rod is at least 4.2 mm. According to some embodiments, the diameter of the rod is at least 4.3 mm. According to some embodiments, the diameter of the rod is at least 4.4 mm. According to some embodiments, the diameter of the rod is at least 4.5 mm. According to some embodiments, the diameter of the rod is at least 4.6 mm. According to some embodiments, the diameter of the rod is at least 4.7 mm. According to some embodiments, the diameter of the rod is at least 4.8 mm. According to some embodiments, the diameter of the rod is at least 4.9 mm. According to some embodiments, the diameter of the rod is at least 5.0 mm

According to some embodiments, the length of the rod ranges from 1.0 cm to 5.0 cm. According to some embodiments, the diameter of the rod is at least 1.0 cm. According to some embodiments, the diameter of the rod is at least 1.1 cm. According to some embodiments, the diameter of the rod is at least 1.2 cm. According to some embodiments, the diameter of the rod is at least 1.3 cm. According to some embodiments, the diameter of the rod is at least 1.4 cm. According to some embodiments, the diameter of the rod is at least 1.5 cm. According to some embodiments, the diameter of the rod is at least 1.6 cm. According to some embodiments, the diameter of the rod is at least 1.7 cm. According to some embodiments, the diameter of the rod is at least 1.8 cm. According to some embodiments, the diameter of the rod is at least 1.9 cm. According to some embodiments, the diameter of the rod is at least 2.0 cm. According to some embodiments, the diameter of the rod is at least 2.0 cm. According to some embodiments, the diameter of the rod is at least 2.1 cm. According to some embodiments, the diameter of the rod is at least 2.2 cm. According to some embodiments, the diameter of the rod is at least 2.3 cm. According to some embodiments, the diameter of the rod is at least 2.4 cm. According to some embodiments, the diameter of the rod is at least 2.5 cm. According to some embodiments, the diameter of the rod is at least 2.6 cm. According to some embodiments, the diameter of the rod is at least 2.7 cm. According to some embodiments, the diameter of the rod is at least 2.8 cm. According to some embodiments, the diameter of the rod is at least 2.9 cm. According to some embodiments, the diameter of the rod is at least 3.0 cm. According to some embodiments, the diameter of the rod is at least 3.1 cm. According to some embodiments, the diameter of the rod is at least 3.2 cm. According to some embodiments, the diameter of the rod is at least 3.3 cm. According to some embodiments, the diameter of the rod is at least 3.4 cm. According to some embodiments, the diameter of the rod is at least 3.5 cm. According to some embodiments, the diameter of the rod is at least 3.6 cm. According to some embodiments, the diameter of the rod is at least 3.7 cm. According to some embodiments, the diameter of the rod is at least 3.8 cm. According to some embodiments, the diameter of the rod is at least 3.9 cm. According to some embodiments, the diameter of the rod is at least 4.0 cm. According to some embodiments, the diameter of the rod is at least 4.1 cm. According to some embodiments, the diameter of the rod is at least 4.2 cm. According to some embodiments, the diameter of the rod is at least 4.3 cm. According to some embodiments, the diameter of the rod is at least 4.4 cm. According to some embodiments, the diameter of the rod is at least 4.5 cm. According to some embodiments, the diameter of the rod is at least 4.6 cm. According to some embodiments, the diameter of the rod is at least 4.7 cm. According to some embodiments, the diameter of the rod is at least 4.8 cm. According to some embodiments, the diameter of the rod is at least 4.9 cm. According to some embodiments, the diameter of the rod is at least 5.0 cm. It should be understood that, according to some embodiments, the diameter of the rod ranges from 0.1 cm to 1.0 cm, and according to other embodiments, the diameter of the rod ranges from 5 cm to 50 cm.

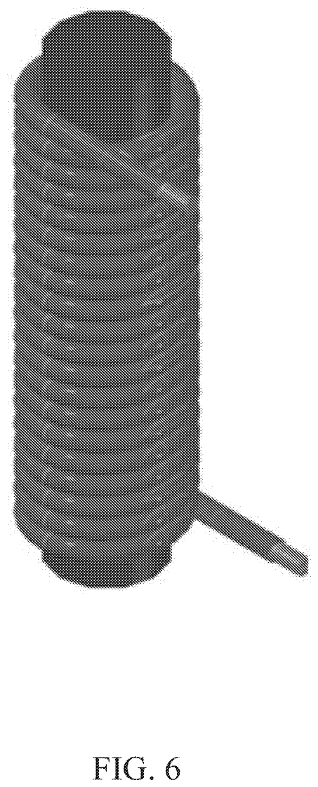

An exemplary and non-limiting example of a stent retrieval tool using a steel rod will now be discussed. A steel rod has magnetic permeability value of 1.03 Tesla. A Tesla is a unit of magnetic induction or magnetic flux density in the meter-kilogram-second system of physical units. Further, the permeability value combined with the cost of steel makes it an adequate option for the retrieval tool. To produce the electromagnet, copper wire is wound down the long axis of the rod, as seen in FIG. 6. An electric current is then applied to the copper wire to increase the magnetic properties of the steel core, thus creating an electromagnet. To supply the power, a circuit can be used to produce an appropriate voltage and/or electric current to magnetize the steel rod. In one example, the power circuit can consist of a direct current power source, (e.g., AAA battery, AA battery, 9 volt battery, button battery, etc.) connected to a resistor to produce a desired electrical current. For example, a 1.5 volt battery connected to a 0.3k Ohm resistor will output approximately 5 mA to the electromagnet, producing the acceptable magnetic field for stent removal. In another example, the power circuit can consist of an alternating power source connected to a transformer and other components to produce the desired electrical current.

It is noted that for patient safety, the electrical current produced by a circuit to power the electromagnet is recommended to not exceed approximately 5 mA (milliamperes). This is because an electrical current greater than 5 mA can cause muscle spasms, paralysis, and even cardiac arrest possibly leading to death (“The Fatal Current.” The Case for Stronger Antibiotic Regulation).

The physician can use an imaging device (e.g., X-ray device, ultrasound device, etc.) to aid the tool in making contact with the proximal end 302. In one embodiment, the stent retrieval tool can comprise an endoscope or any other camera type device. The endoscope can be used to aid a physician in guiding the stent retrieval tool to make contact with the proximal end 302.

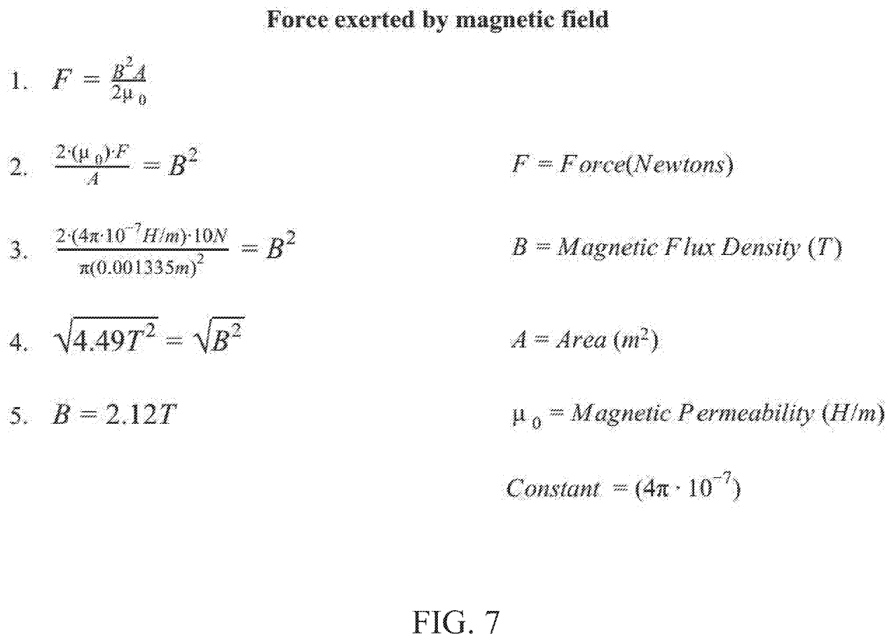

To remove the ureteral stent device, a magnetic pull force of approximately 5 Newtons is recommended. (Wang, Jin, et al. “Preclinical Evaluation of a Newly Designed Ureteral Stent and Magnetic Retrieval Catheter for Minimally Invasive Stent Removal.” Urology, vol. 84, no. 4, 2014, pp. 960-966, doi:10.1016/j.urology.2014.06.024). Magnetization is often characterized by the magnetic flux density. The magnetic flux is a measurement of the total magnetic field which passes through a given area. Identifying the magnetic flux density of the stent retrieval device can determine its dimensions. FIG. 7 shows an equation (Equation 1) to determine a force exerted by the magnetic field. The force, magnetic permeability, and the cross-section area are known, and the equation is solved (Equations 2-5) for the magnetic flux density. By way of example for a neodymium magnet, the magnetic flux density is 2.12 Tesla. It should be noted that as the cross section area decreases, the magnetic flux density increases. As such, those skilled in the art would be able to use Equation 1 to adjust the ureteral stent device to a desired magnetic flux density and cross area.

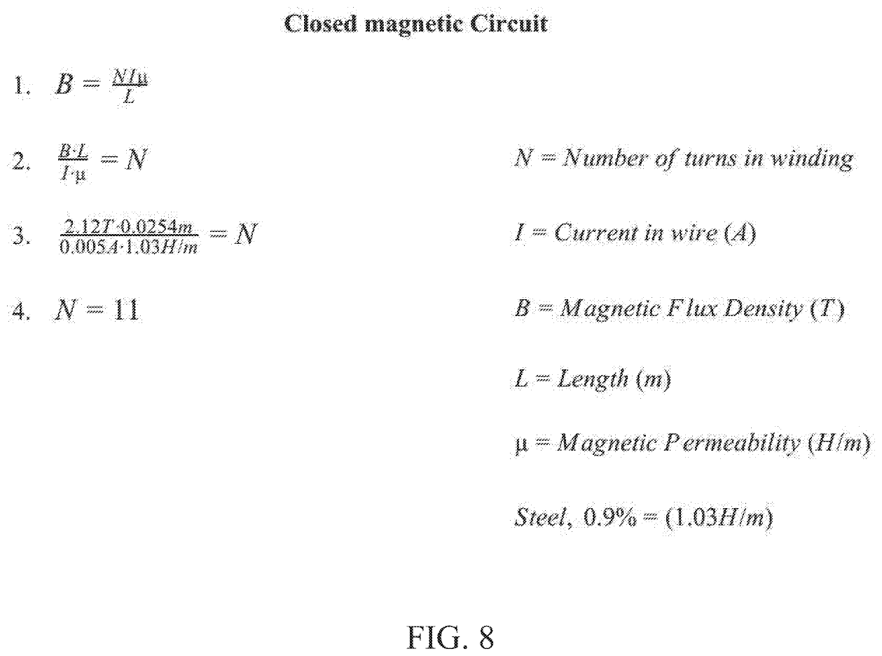

FIG. 8 shows an equation (Equation 1) to adjust the dimensions of the electromagnet. In this example, the magnetic flux density, electrical current, permeability of steel, and length are all known values. Therefore, Equation 6 can be rearranged to solve for N, which is the number of turns, as seen in Equations 7-9. The number of turns determined in this example is 11 (Equation 2). It is understood by those skilled in the art that as electrical current decreases, the number of turns increases.

Experimental results of using the ureteral stent device 300 and using the stent retrieval tool will now be discussed. It is noted that these results are discussed by way of example for illustrative purposes, and are not limiting to the invention discussed herein. In the experiment, the tensile strength required to remove the ureteral stent device 300 using the stent retrieval tool was tested via a Bose ElectroForce 3200 load frame system. The flow of urine through the ureteral stent device 300 was calculated by pumping water at 40° C., which is theoretically equivalent to the viscosity of urine, through the ureteral stent device 300 while a pressure gauge measured inlet and outlet pressures, using Equation 10, below:

$$\frac{Q * 128 µ * L}{π * {D}^{4}} + {P}_{in} = {P}_{out} \qquad Equation 10$$

Where:
Q=Flow rate [m³/sec],
Poiseuille Flow Constant=128,
μ=viscosity of liquid [Pa*sec]
L=length of the ureteral stent device 300 [m],
D=inner diameter of the ureteral stent device 300 [m],
P in =Pressure at the inlet [Pa],
P_{ow}=Pressure at outlet [Pa]

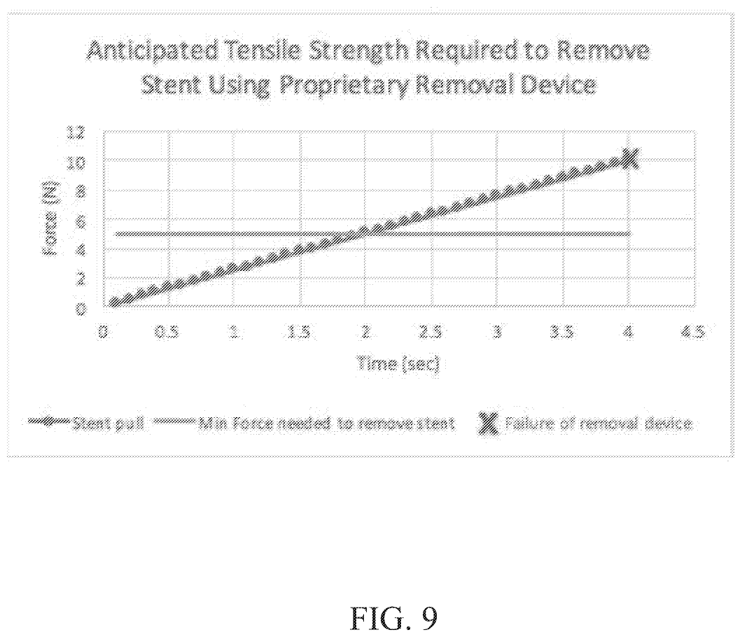

FIG. 9 is a graph showing the force required to separate the ureteral stent device 300 and the stent retrieval tool, and the flow rate of “urine” through the ureteral stent device 300. As shown, the ureteral stent device 300 and the stent retrieval tool separate at 10 Newtons (“N”) of force. A Newton, the SI unit of force, is the amount of force required to make a mass of one kilogram accelerate at a rate of one meter/second². Any value less than 5 N would result in failure to remove the stent from the ureter. This provides a factor of safety of two.

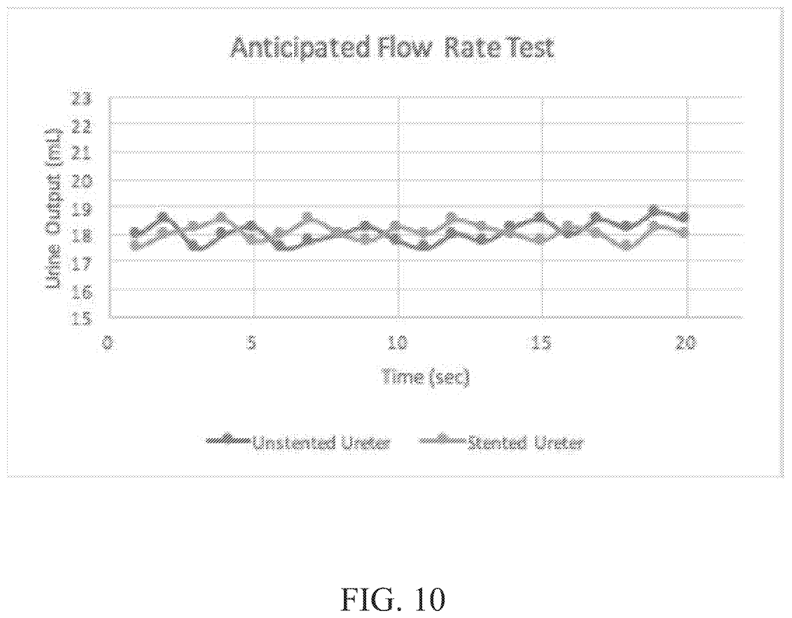

FIG. 10 is a graph showing projected results for a flow rate test using the ureteral stent device 300. Specifically, FIG. 10 shows that urine flow (meaning moving along in a steady, continuous stream) using the ureteral stent device 300 is comparable to normal, healthy urine flow (e.g., the urine flow rate of an unstented, unrestricted ureter), at a flow rate between 17.5 and 18.75 ml/sec. It is noted that inability to maintain normal flow rate of urine has the potential to cause kidney damage.

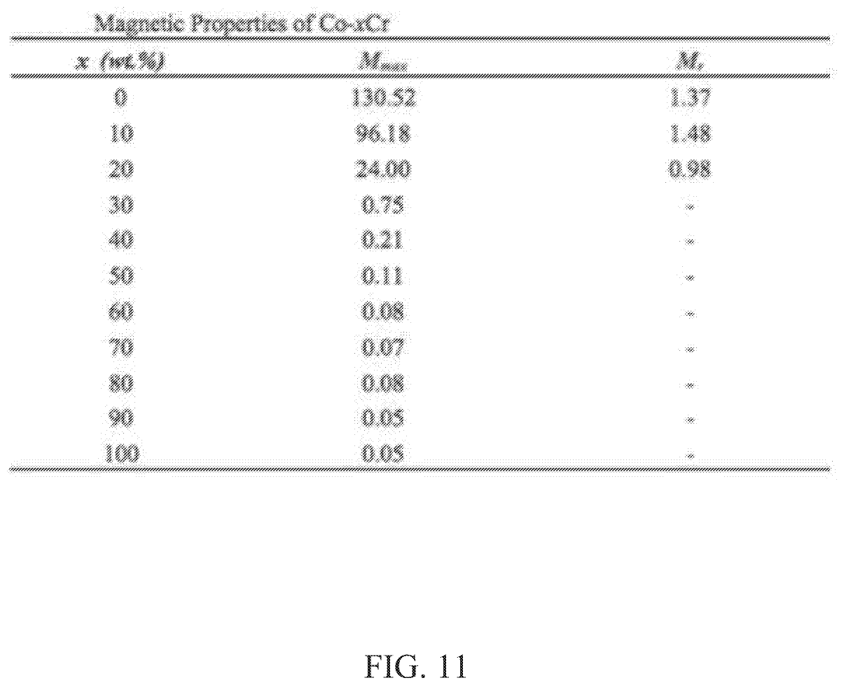

It should be understood that the exemplary results and experiments discussed above relate to the proximal end being a neodymium magnet. However, as discussed above, other materials (e.g., magnets, metals, alloys, etc.) can be used for the proximal end 302. It should be understood that the material should have biocompatible and corrosion resistant properties due to its clinical use. The material should also contain magnetic properties, although a material with high magnetic flux density and magnetic remanence may interfere with the fields produced by a MRI. By way of example, cobalt-chromium is another suitable material to be used as the proximal end. Cobalt is a ferromagnetic material that has a magnetic flux density of 0.5 T. Ferromagnetic materials are materials that can be magnetized by an external magnetic field. The magnetic flux should be less than 0.5 T to be considered MRI safe, therefore another material should be added to cobalt to reduce the flux. (How Strong Are the Magnets in an MRI Machine?” HowStuffWorks Science, HowStuffWorks, 28 Jun. 2018.) Chromium is a paramagnetic materials, so it cannot be magnetized by external magnetic field. A certain alloy wt % (means weight percent) of cobalt and chromium would keep the magnetic properties of cobalt. FIG. 11 shows that under 20% chromium, the alloy would stay magnetic.

Neodymium (Nd) was selected for prototyping and proof of concept because it has magnetic properties, is inexpensive, and is easily attainable with the given size constraints and parameters. However, Neodymium has three times greater the magnetic flux density than cobalt-chromium. (“Magnet Tables & Demagnetization Curves.” SuperMagnetMan, supermagnetman.com/pages/tables-curves). To compensate for the difference, the stent retrieval device can have a higher magnetic field, which is accomplished by increasing the number of turns.

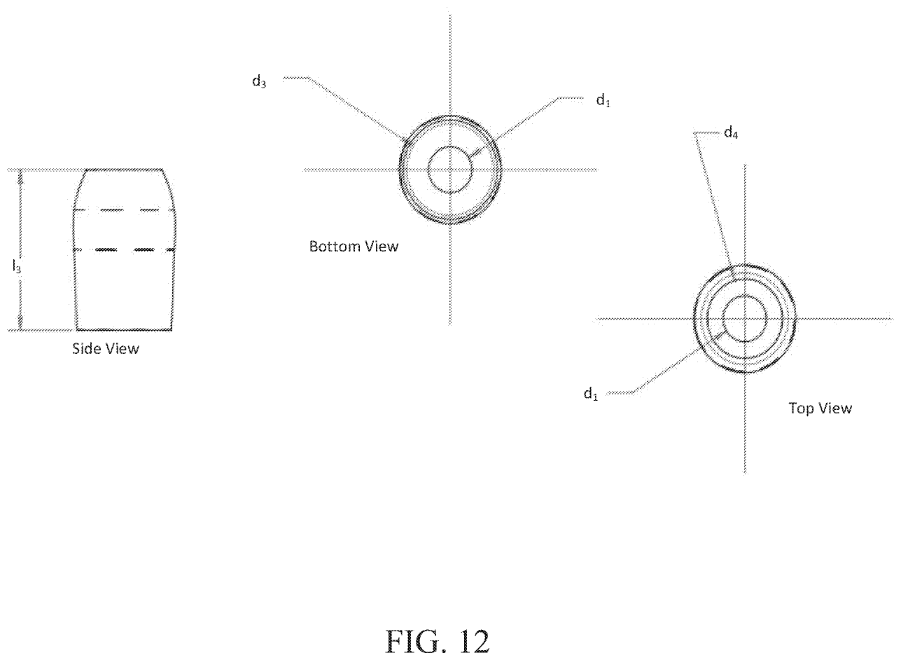

FIG. 12 shows an exemplary and non-limiting example of a drawing for a cobalt-chromium proximal end. The drawings shows length l₃, and diameters d₁, d₃, and d₄. Ranges and values for length l₃ and diameters d₁ and d₃ have been discussed above. According to some embodiments, d₄ ranges from 1.0 mm to 5.0 mm. According to some embodiments, d₄ is at least 1.0 mm. According to some embodiments, d₄ is at least 1.1 mm. According to some embodiments, d₄ is at least 1.2 mm. According to some embodiments, d₄ is at least 1.3 mm. According to some embodiments, d₄ is at least 1.4 mm. According to some embodiments, d₄ is at least 1.5 mm. According to some embodiments, d₄ is at least 1.6 mm. According to some embodiments, d₄ is at least 1.7 mm. According to some embodiments, d₄ is at least 1.8 mm. According to some embodiments, d₄ is at least 1.9 mm. According to some embodiments, d₄ is at least 2.0 mm. According to some embodiments, d₄ is at least 2.0 mm. According to some embodiments, d₄ is at least 2.1 mm. According to some embodiments, d₄ is at least 2.2 mm. According to some embodiments, d₄ is at least 2.3 mm. According to some embodiments, d₄ is at least 2.4 mm. According to some embodiments, d₄ is at least 2.5 mm. According to some embodiments, d₄ is at least 2.6 mm. According to some embodiments, d₄ is at least 2.7 mm. According to some embodiments, d₄ is at least 2.8 mm. According to some embodiments, d₄ is at least 2.9 mm. According to some embodiments, d₄ is at least 3.0 mm. According to some embodiments, d₄ is at least 3.1 mm. According to some embodiments, d₄ is at least 3.2 mm. According to some embodiments, d₄ is at least 3.3 mm. According to some embodiments, d₄ is at least 3.4 mm. According to some embodiments, d₄ is at least 3.5 mm. According to some embodiments, d₄ is at least 3.6 mm. According to some embodiments, d₄ is at least 3.7 mm. According to some embodiments, d₄ is at least 3.8 mm. According to some embodiments, d₄ is at least 3.9 mm. According to some embodiments, d₄ is at least 4.0 mm. According to some embodiments, d₄ is at least 4.1 mm. According to some embodiments, d₄ is at least 4.2 mm. According to some embodiments, d₄ is at least 4.3 mm. According to some embodiments, d₄ is at least 4.4 mm. According to some embodiments, d₄ is at least 4.5 mm. According to some embodiments, d₄ is at least 4.6 mm. According to some embodiments, d₄ is at least 4.7 mm. According to some embodiments, d₄ is at least 4.8 mm. According to some embodiments, d₄ is at least 4.9 mm. According to some embodiments, d₄ is at least 5.0 mm.

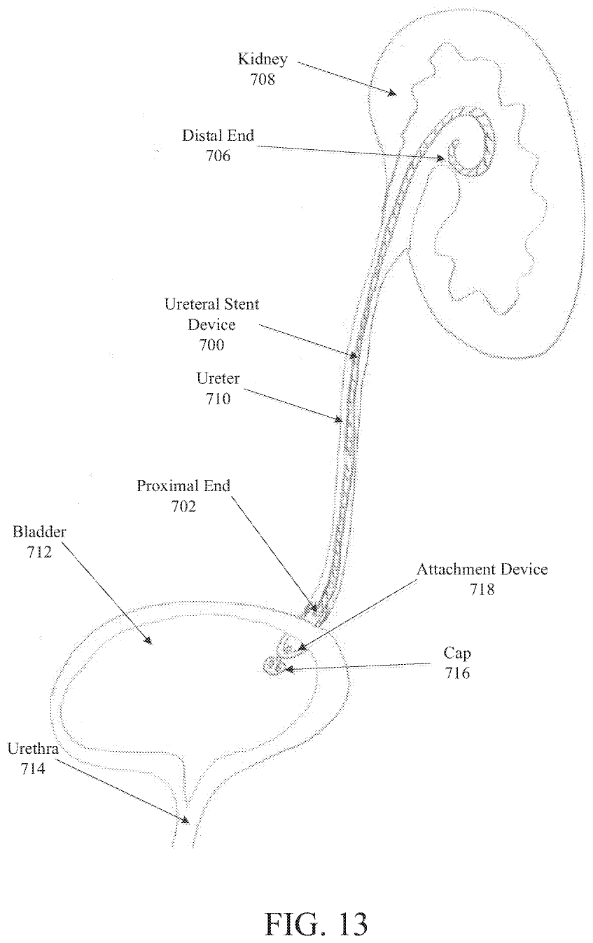

FIG. 13 shows an exemplary and non-limiting example of an ureteral stent device 700 of the described invention. The ureteral stent device 700 comprises a proximal end 702, a stent body 704, a distal end 706, a cap 716, and an attachment device 718. FIG. 13 further shows a kidney 708, a ureter 710, a bladder 712, and a urethra 714. The stent body 704 comprises a tubular structure comprising an inner diameter (d₁) and an outer diameter (d₂) (ranges and values for d₁ and d₂ have been discussed above), making the stent body 704 hollow. The hollow body allows urine to flow through the ureteral stent device 700. However, those skilled in the art would understand that the inner diameter can be any length necessary to allow for a healthy urine flow rate, and the outer diameter can any length to allow the ureteral stent device 700 to be placed into the ureteral 310.

The cap 716 is attached to the proximal end 702 via the attachment device 718. The attachment device 718 can be a string, a flexible polymer, or any other type of material suitable to attach the cap 716 to the proximal end 702. The attachment device 718 can be coated to reduce encrustation in the bladder. By way of example, the coating can comprise heparin, phosphorylcholine, antibiotic, carbon, hyaluronic acid, triclosan, silver, gendine, chitosan, salicylic acid, hydrogel, Teflon, titanium, or any other suitable material effective to reduce encrustation. The cap 716 comprises a porous-dome shape, wherein the shape prevents the ureteral stent device 700 from receding into the ureter 710. The shape further allows for a retrieval tool to be attached to the pores in the cap 716, and to remove the ureteral stent device 700 from the body via the urethra.

According to some embodiments, the length of the cap 716 ranges from 1.0 mm to 5.0 mm. According to some embodiments, the length of the cap 716 is at least 1.0 mm. According to some embodiments, the length of the cap 716 is at least 1.25 mm. According to some embodiments, the length of the cap 716 is at least 1.5 mm. According to some embodiments, the length of the cap 716 is at least 1.75 mm. According to some embodiments, the length of the cap 716 is at least 2.0 mm. According to some embodiments, the length of the cap 716 is at least 2.25 mm. According to some embodiments, the length of the cap 716 is at least 2.5 mm. According to some embodiments, the length of the cap 716 is at least 2.75 mm. According to some embodiments, the length of the cap 716 is at least 3.0 mm. According to some embodiments, the length of the cap 716 is at least 3.25 mm. According to some embodiments, the length of the cap 716 is at least 3.5 mm. According to some embodiments, the length of the cap 716 is at least 3.75 mm. According to some embodiments, the length of the cap 716 is at least 4.0 mm. According to some embodiments, the length of the cap 716 is at least 4.25 mm. According to some embodiments, the length of the cap 716 is at least 4.75 mm. According to some embodiments, the length of the cap 716 is at least 5.0 mm. According to some embodiments, the length of the cap 716 is at least 5.25 mm. According to some embodiments, the length of the cap 716 is at least 5.5 mm. According to some embodiments, the length of the cap 716 is at least 5.75 mm. According to some embodiments, the length of the cap 716 is at least 6.0 mm. According to some embodiments, the length of the cap 716 is at least 6.25 mm. According to some embodiments, the length of the cap 716 is at least 6.5 mm. According to some embodiments, the length of the cap 716 is at least 6.75 mm. According to some embodiments, the length of the cap 716 is at least 7.0 mm. According to some embodiments, the length of the cap 716 is at least 7.25 mm. According to some embodiments, the length of the cap 716 is at least 7.5 mm. According to some embodiments, the length of the cap 716 is at least 7.75 mm. According to some embodiments, the length of the cap 716 is at least 8.0 mm. According to some embodiments, the length of the cap 716 is at least 8.25 mm. According to some embodiments, the length of the cap 716 is at least 8.5 mm. According to some embodiments, the length of the cap 716 is at least 8.75 mm. According to some embodiments, the length of the cap 716 is at least 9.0 mm. According to some embodiments, the length of the cap 716 is at least 9.25 mm. According to some embodiments, the length of the cap 716 is at least 9.5 mm. According to some embodiments, the length of the cap 716 is at least 9.75 mm. According to some embodiments, the length of the cap 716 is at least 10.0 mm. According to some embodiment, the length of the cap 716 is greater than 10.0 mm

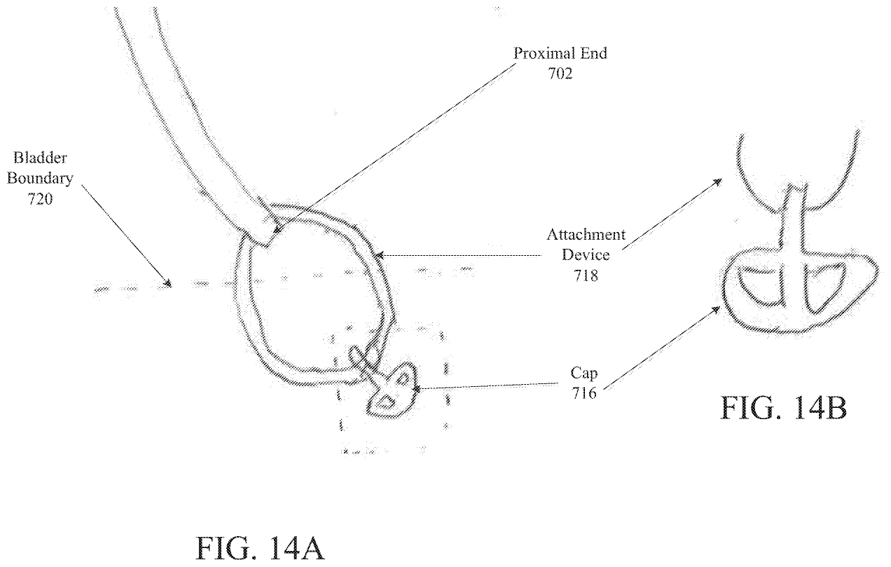

FIGS. 14A and 14B show a closer view of the cap 716 and the attachment device 718. The bladder boundary 720 is a representative line for illustration purposes of the location of the bladder entrance in relation to the attachment device 718. Those skilled in the art would understand that the location of the attachment device 718 in FIG. 14A is by way of example.

Returning to FIG. 13, the distal end 706 can comprise a tip and can form any one of a J-curl, a j-shape, a pigtail loop, or any other type of curl capable of securing the distal end 706 in the kidney 708. The tip can be tapered. The kidney 708 is used to show a general relation between the kidney entrance and the distal end 706, and is used for illustrative purposes only. Similarity, the bladder 712 is used to show a general relation between the bladder 712 and the proximal end 702, the cap 716, and the attachment device 718, and is used for illustrative purposes only.

In this embodiment, the proximal end 702 does not enter the bladder 712, and terminates in the ureter 710, the cap 716 is positioned inside the bladder 712, and the attachment device 718 is positioned partly in the ureter 710 and partly in the bladder 712. This is solely by way of example. In other embodiments, the proximal end 702 can terminal in the bladder 712, or anywhere else along the ureter 710.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and described the methods and/or materials in connection with which the publications are cited.

It must be noted that as used herein and in the appended claims, the singular forms “a”, “an”, and “the” include plural references unless the context clearly dictates otherwise.

The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A ureteral stent device, comprising:
a tubular structure comprising a stent body, a proximal end and a distal end, wherein:
the stent body comprises a first diameter and a second diameter forming a hollow tubular structure wherein the second diameter is greater than the first diameter;
the distal end comprises a tip capable of forming a curl for securing the distal end in a kidney;
the proximal end comprises a third diameter and a material having magnetic properties; and
the proximal end terminates in a ureter.

2. The ureteral stent device of claim 1, wherein the proximal end comprises at least one of a magnet, a magnetic tip, a magnetic coating, a magnetic metal or a magnetic alloy.

3. The ureteral stent device of claim 1, wherein the proximal end comprises at least one of a neodymium iron baron magnet, cobalt-chromium, or a biocompatible magnet.

4. The ureteral stent device of claim 1, wherein the distal end is tapered.

5. The ureteral stent device of claim 1, wherein the proximal end is coated in a biocompatible coating capable of reducing encrustation.

6. The ureteral stent device of claim 5, wherein the coating comprises at least one of Teflon, titanium, heparin, phosphorylcholine, antibiotic, carbon, hyaluronic acid, triclosan, silver, gendine, chitosan, salicylic acid, or hydrogel.

7. The ureteral stent device of claim 1, wherein the proximal end is fixated to the stent body by at least one of a waterproof adhesive, polyurethane glue, a clip mechanism, a screwing mechanism, welding, soldering, taping, or inserting.

8. The ureteral stent device of claim 1, wherein the third diameter is greater or equal to the second diameter.

9. The ureteral stent device of claim 1, wherein the ureteral stent device is retrieved from the ureter with a stent retrieval tool, the stent retrieval device comprising a first end capable of making contact with the proximal end of the ureteral stent device.

10. The ureteral stent device of claim 9, wherein the stent retrieval tool comprises a magnet at the first end.

11. The ureteral stent device of claim 10, wherein the magnet comprises a coating capable of reducing encrustation.

12. The ureteral stent device of claim 11, wherein the coating comprises at least one of Teflon, titanium, heparin, phosphorylcholine, antibiotic, carbon, hyaluronic acid, triclosan, silver, gendine, chitosan, salicylic acid, or hydrogel.

13. The ureteral stent device of claim 9, wherein the stent retrieval tool comprises a rod at the first end.

14. The ureteral stent device of claim 13, wherein the rod comprises at least one of steel, stainless steel, iron, nickel, cobalt, cobalt-chromium, rare earth metals or a rod capable of being magnetized.

15. The ureteral stent device of claim 13, wherein the rod is electromagnetically charged and contacted to the proximal end of the ureteral stent device to remove ureteral stent the device from the ureter.

16. The ureteral stent device of claim 9, wherein the first end comprises a fourth diameter, wherein the fourth diameter is less than or equal to the third diameter.

17. A ureteral stent device, comprising:
a tubular structure comprising a stent body, a proximal end, a distal end, a cap and an attachment device, wherein:
the stent body comprises an first diameter and a second diameter forming a hollow tubular structure hollow where the second diameter is greater than the first diameter;
the distal end comprises a tip capable of forming a curl for securing the distal end in a kidney;
the proximal end is attached to the cap via the attachment device;
the proximal end terminates in a ureter; and
at least a part of the cap is situated in a bladder.

18. The ureteral stent device of claim 17, wherein the attachment device comprises one of a string or a flexible polymer.

19. The ureteral stent device of claim 17, wherein the cap is coated in a biocompatible coating capable of reducing encrustation.

20. The ureteral stent device of claim 19, wherein the coating comprises at least one of Teflon, titanium, heparin, phosphorylcholine, antibiotic, carbon, hyaluronic acid, triclosan, silver, gendine, chitosan, salicylic acid, or hydrogel.

21. The ureteral stent device of claim 17, wherein the cap comprises a porous-dome shape.

22. The ureteral stent device of claim 21, wherein the shape allows for a retrieval tool to be attached to pores in the cap and to remove the ureteral stent device from the ureter via the urethra.

23. The ureteral stent device of claim 17, wherein the cap is prevents the ureteral stent device from receding into the ureter.
